(19) United States
(12) Patent Application Publication
(10) Pub. No.: US 2026/0263808 A1
(43) Pub. Date: Sep. 10, 2026

(54) DEPTH STEREO-ELECTROENCEPHALOGRAPHY ELECTRODE FOR INTRACRANIAL NEURONAL ELECTRICAL STIMULATION OF THE BRAIN

(71) Applicant: Vasileios KOKKINOS, Lamia (GR)

(72) Inventor: Vasileios KOKKINOS, Lamia (GR)

(21) Appl. No.: 19/158,003

(22) PCT Filed: Sep. 8, 2023
(86) PCT No.: PCT/EP2023/074690

§ 371 Date: Aug. 19, 2025

(30) Foreign Application Priority Data
Feb. 23, 2023 (GR) 20230100157

(51) Int. Cl. A61N 1/36; A61N 1/05

(52) U.S. Cl. CPC A61N 1/36135; A61N 1/0534; A61N 1/36064; A61N 1/36142

(57) ABSTRACT

Depth sEEG electrode for intracranial neural electrical stimulation of the brain, comprising temperature sensors to protect the brain tissue from damage due to unwanted local increase in temperature as a result of sustained/chronic intracranial neural electrical stimulation, and further comprising moisture sensors to detect deviations from baseline intra-parenchymal liquid volume and/or pressure values. If, during stimulation through the electrode, one or more of the temperature values rise above and outside the predefined safety limits, the neurostimulation/sEEG device can immediately interrupt the ongoing neurostimulation, in order to protect the brain tissue from undesirable thermal side effects. The liquid volume and/or pressure values detected can be conveyed to the neurostimulation/sEEG device, which in turn displays them to the physician/neurosurgeon responsible for the sEEG procedure, to assess whether the implanted electrode resides in its predetermined location or resides partially or fully in cerebrospinal fluid space inside or outside the brain parenchyma, as well as to assess the risk of intracranial hemorrhage.

### TECHNICAL FIELD

The present invention relates to a type of depth electrode for intracranial neuronal electrical stimulation of the brain, enhanced with temperature sensors to protect brain tissue from damage due to unwanted local temperature increase as a result of continuous/chronic electrical stimulation, and moisture sensors to detect contacts residing in cerebrospinal fluid (CSF) space inside or outside the brain parenchyma, as well as potential subdural or intra-parenchymal hemorrhage.

### BACKGROUND

Epilepsy is a devastating brain disorder that affects nearly 50 million people worldwide. Epilepsy is the fourth most common neurological disorder and is manifested by seizures. Epilepsy is defined as a “sudden, excessive, and rapid discharge” of neuronal populations in the brain, detected by the electroencephalogram (EEG). Seizures have significant and often devastating consequences for patients' quality of life, excluding them from common activities (such as driving, swimming, etc.), affecting their personal life and family planning, as well as stigmatizing them socially and professionally. In addition, poorly controlled seizures can cause harmful conditions of varying severity, ranging from prolonged seizures leading to the condition known as status epilepticus to seizures followed by severe cardiorespiratory arrest, either during or after their end, which are responsible for the “sudden, unexpected death in epilepsy-SUDEP”.

Today, two main therapeutic pathways are widely available: antiepileptic drugs and epilepsy surgery. Treatment of epilepsy with antiepileptic drugs achieves seizure-freedom in 70% of patients. Nevertheless, the side effects of antiepileptic drugs can have significant consequences on the patients' quality of life, resulting in the failure of therapeutic goals in 40% of patients. Epilepsy surgery is an established treatment option for drug-resistant patients. The main goal of epilepsy surgery is to completely resect (or completely disconnect) areas of the brain responsible for the primary organization of epileptic activity that gives rise to seizures. This procedure often includes a diagnostic phase, in which depth electrodes are implanted into the brain parenchyma in order to locate the area of the brain responsible for producing seizures, the epileptogenic zone. During this phase, where the patient remains and is recorded in the specially configured Epilepsy Monitoring Unit (EMU), intracranial neuronal electrical stimulation is often performed for two reasons: 1. To map areas that serve basic brain functions (movement, sensation, vision, hearing, speech, memory), so that they can be excluded from any surgical intervention, and 2. For the mapping of the epileptogenic zone, which will be the main surgical target.

Nevertheless, it is estimated that only 50% of patients with drug-resistant epilepsy become candidates for epilepsy surgery. For those patients who are “refractory” to both antiepileptic therapy and epilepsy surgery, intracranial neuronal electrical stimulation (or neurostimulation) is offered as a palliative treatment to improve seizure control. Over the past few decades, the US Food and Drug Administration (FDA) has approved neurostimulation devices to address both treatment failures of antiepileptic drugs and failures to enroll patients in a surgical treatment plan. In particular, both open-loop (i.e. systems that do not have the possibility of data feedback), such as deep brain stimulation (DBS), but also closed-loop (i.e. systems that include the possibility of data feedback) neuronal electrical stimulation systems have been approved and are being applied clinically, such as responsive neurostimulation (RNS).

Both in the context of the diagnostic pre-therapeutic stereo-electroencephalography (sEEG) phase, as well as in the context of the therapeutic phase, intracranial neuronal electrical stimulation is an important part of the procedures and is carried out through specially designed depth sEEG electrodes. When applying intracranial neuronal electrical stimulation, a local electrical field is created between the two contacts that are selected, which disrupts the electrical activity of the adjacent brain tissue. And while, for both diagnostic and therapeutic reasons, current depth sEEG electrode implementations have placed great emphasis on the quality and reliability of the electrical stimulation, they have not addressed the thermal effects that develop in the adjacent brain tissue as a result (or side effect) of the application of a local electric field of a prolonged and/or chronic nature during the execution of the electrical stimulation. It is known that increases in temperature beyond safety limits in brain tissue, whether local or generalized, are associated with abrupt or early degradation of neuronal cells, disruption of local vascular structure, and the consequent development of brain lesions. In addition, prior art depth sEEG electrode designs have not addressed the problem of potential mislocations of the depth electrodes, ending up fully or partially in intra-parenchymal or extra-parenchymal spaces filled with cerebrospinal fluid (CSF), or rupturing vessels along their trajectory and causing subdural or intra-parenchymal hemorrhage. A need therefore arises to develop depth sEEG electrodes for intracranial neuronal electrical stimulation with which sustained/chronic electrical stimulation can be provided in a safe manner when epileptic patients undergo intracranial diagnostic and/or therapeutic procedures.

### SUMMARY OF THE INVENTION

According to the present invention, a type of depth sEEG electrode for intracranial neuronal electrical stimulation of the brain is provided, enhanced with one or more temperature sensors to protect brain tissue from damage due to unwanted local temperature increase as a result of sustained/chronic electrical stimulation, and one or more moisture sensors to determine deviations from baseline intra-parenchymal liquid volume and/or pressure values in any of the moisture sensors according to claim 1.

According to the present invention, a depth sEEG electrode configured to be implanted into the brain parenchyma is provided, said electrode comprising:
a series of electrical contacts along the length of the electrode configured to provide intracranial neuronal electrical stimulation;
one or more temperature sensors interposed between one or more electrical contacts, wherein each temperature sensor is adapted to detect the temperature values generated in the brain parenchyma by the generated electrical field as a result of prolonged and/or chronic intracranial neuronal electrical stimulation; and
one or more moisture sensors, wherein each moisture sensor is adapted to detect the liquid volume and/or pressure values at the site of implantation, in order to detect deviations from baseline intra-parenchymal liquid volume and/or pressure values.

An important advantage of said electrode is that it may be used to provide electrical neurostimulation in a safer and more efficient manner. For instance, during electrical neurostimulation, when one or more of the integrated temperature sensors detect that the temperature rises above predetermined safety limits, this information may be used to immediately interrupt the intracranial neuronal electrical stimulation. In addition, the integrated moisture sensors help determine whether a potentially life-threatening intracranial hemorrhage, such as subdural or intra-parenchymal hemorrhage, has occurred during the placement of the electrode. Also, the integrated moisture sensors can detect early enough electrode mislocations that greatly reduce the efficiency of the applied neurostimulation and sEEG recording. For instance, the liquid volume and/or pressure values may indicate that one or more contacts reside in cerebrospinal fluid space inside or outside the brain parenchyma.

In a preferred embodiment, the temperature value detected by the one or more temperature sensors is transferred to an external electrical device, preferably an external electrical neurostimulation device. When the temperature value transferred by the one or more temperature sensors rises above predetermined safety limits, the external device immediately interrupts the intracranial neuronal electrical stimulation. In this way, optimal control of intracranial neuronal electrical stimulation is achieved through detection of the temperature developed between adjacent electrical contacts, in order to protect the brain parenchyma from undesirable thermal phenomena.

In another preferred embodiment, the liquid volume and/or pressure values detected by the one or more moisture sensors are transferred to an external electrical device, that can be either an external electrical neurostimulation or an sEEG device, in order to evaluate the liquid volume and/or pressure values at the site of implantation and assess the location of the implanted electrode or detect a potential subdural or intra-parenchymal hemorrhage.

In a most preferred embodiment, the temperature values and the liquid volume and/or pressure values detected are transferred to the external electrical device, such that, if the temperature values are elevated beyond predetermined safety limits, the external device immediately interrupts the intracranial neuronal electrical stimulation; if the liquid volume and/or pressure values from one or more moisture sensors indicate that one or more contacts reside in cerebrospinal fluid space inside or outside the brain parenchyma, the electrode is removed for re-implantation; and if the liquid volume and/or pressure values from one or more moisture sensors indicate a potential subdural or intra-parenchymal hemorrhage, early action to stop the hemorrhage can be taken.

Further features and advantages of the present invention, as well as the structure and operation of various embodiments of the present invention, are described in detail below with reference to the accompanying drawings.

### BRIEF DESCRIPTION OF THE DRAWINGS

The present invention will now be described with reference to certain embodiments thereof illustrated in the accompanying drawings. It should be noted that the attached drawings illustrate preferred embodiments of the invention, therefore they should not be considered as limiting the scope of the invention. It is also understood that the illustrations may include optional features which are not necessary in any embodiment.

FIG. 1 schematically illustrates a cross-sectional side view of a typical prior art depth sEEG electrode (2) implanted within the brain parenchyma (1) as well as the local electric fields (4) generated between adjacent electrical contacts (3) during its intracranial neuronal electrical stimulation.

FIG. 2 schematically illustrates a cross-sectional side view of the proposed enhanced type of depth sEEG electrode (8), where temperature sensors (5) are placed between standard electrical contacts (3), and moisture sensors (12) are placed on either side of each electrical contact (3), as well as their corresponding internal wiring (6, 7 and 13 respectively).

FIG. 3 schematically illustrates a possible implementation of the proposed enhanced depth sEEG electrode (8), in the context of its use by a hypothetical neurostimulation or sEEG device, through an electronic system that would interrupt the conduct of intracranial neuronal electrical stimulation when one or more temperature sensors (5) register a local increase in temperature above safety limits, and would immediately notify for deviations from baseline intra-parenchymal moisture values in any of the moisture sensors (12).

### DETAILED DESCRIPTION OF THE INVENTION

The invention is described below, with reference to detailed illustrative embodiments. It will be apparent that a system according to the invention can be embodied in a wide variety of forms. Accordingly, the specific structural and functional details disclosed herein are representative and do not limit the scope of the invention.

Terms not specifically defined herein should be given the meaning that would be given to them by one skilled in the art in light of the present disclosure and the general context.

As used herein, the terms “therapeutic” and “treatment” refer to the elimination, reduction, suppression, inhibition of the progression, severity and/or extent of a disease, lesion, clinical sign or symptom in a subject. Said terms also refer to the alleviation, in whole or in part, of the clinical signs and symptoms associated with a disorder or disease such as, for example, epilepsy.

The term “depth sEEG electrode” refers to intracranial electrodes, typically of the needle-like cylindrical type with built-in recording contacts, specially designed to penetrate the brain parenchyma and reach the deeper structures of the brain.

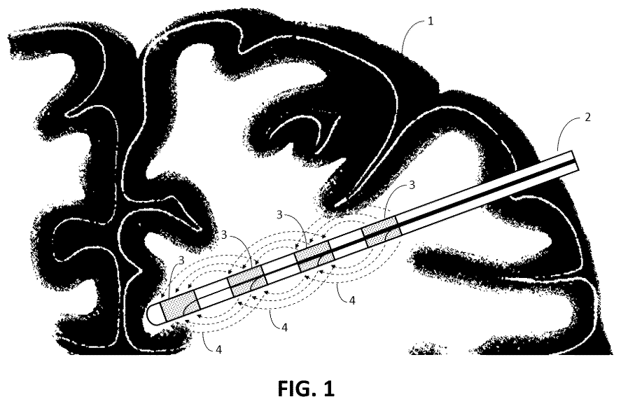

The present invention takes into account the already well-established fact that when intracranial neuronal electrical stimulation is applied between two electrical contacts (3) on a typical prior art depth sEEG electrode (2), a local electrical field (4) is generated between neighboring contacts. Said local electric field created and maintained throughout the intracranial neuronal electrical stimulation is intended to disrupt the electrical activity of the adjacent brain tissue (1) in order to generate functional responses, provoke seizures or act therapeutically in the genesis of epileptic seizures. For both diagnostic and therapeutic reasons, current implementations of depth sEEG electrodes (2), such as the one illustrated in FIG. 1, have placed great emphasis on the quality and reliability of intracranial neuronal electrical stimulation but have not taken into account the thermal effects that develop in the adjacent brain tissue as result of the prolonged and/or chronic application of a local electric field during the electrical stimulation. It is known that temperature increases over and above the safety limits in the brain tissue, whether they are local or generalized, are associated with a variety of neurodegenerative phenomena, such as abrupt or early degradation of neuronal cells, disruption of the structure of local vasculature, which have as a consequence the development of local and/or generalized brain lesions.

The present invention also takes into account the already well-established fact that when depth sEEG electrodes (2) are inserted surgically into the brain, they may deviate from their original planned trajectory, ending up fully or partially in CSF space (inside or outside the brain parenchyma) or colliding with vessels whose injury may cause intracranial hemorrhage. The placement of depth sEEG electrodes (2) in ventricular space is a benign situation, usually caused by incomplete opening of the dura matter, where specific electrode contacts do not record from the brain and intracranial neuronal electrical stimulation cannot be performed due to the high impedance generated by the CSF surrounding specific electrical contacts (3). The placement of depth sEEG electrodes (2) in arachnoid space or extradural space is also a benign situation, usually caused by incomplete or failed opening of the dura matter, where no electrode contact resides in or records from the brain, and subsequently intracranial neuronal electrical stimulation cannot be performed. However, the collision of depth sEEG electrodes with vessels of the brain is a less benign situation, that can cause hemorrhage of variable extend and is considered a potentially life-threatening situation for the patient.

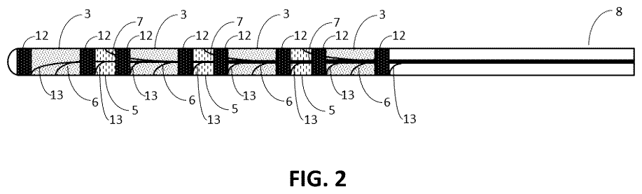

The present invention is based on the original idea that by augmenting typical depth sEEG electrodes (2) for intracranial neuronal electrical stimulation with temperature sensors (5), we can protect brain tissue from damage due to local temperature increase as a result of continuous/chronic electrical stimulation. The proposed depth sEEG electrode (8) includes a series of electrical contacts (3) along the length of the electrode for the performance of intracranial neuronal electrical stimulation (4). The electrode (8) further includes one or more temperature sensors (5), placed in a cylindrical fashion along the length of the electrode and adapted to detect temperature values during the application of intracranial neuronal electrical stimulation (4). In the preferred embodiment illustrated in FIG. 2, a temperature sensor (5) is provided for each contact (3) such that one temperature sensor (5) is placed between two successive contacts (3). In this intermediate position between the electrical contacts, each temperature sensor (5) records the values of the temperature between the respective adjacent electrical contacts (3), which are created by the generated electric field (4) as a result of prolonged and/or chronic intracranial neuronal electrical stimulation in the specific electrical contacts (3). The location of the temperature sensors in the embodiment shown in FIG. 2 allows a) temperature values to be obtained between the specific electrically excited contacts, and b) values to be obtained from all pairs of its contacts. Typically, local temperature increases of more than one degree Celsius during intracranial neuronal electrical stimulation relative to the local temperature value before its onset are considered harmful to the immediately surrounding brain tissue. In an alternative embodiment, a single temperature sensor can be provided for every two contacts, or every three contacts, or any other number of contacts. In a further alternative, one or more of said temperature sensors (5) can be placed outside the area between two successive contacts (3).

The present invention is also based on the original idea that by augmenting typical depth sEEG electrodes (2) with moisture sensors (12), electrode mislocations and potential intracranial hemorrhages can be detected early enough. The proposed depth sEEG electrode (8), as described in the previous paragraph, includes one or more moisture sensors (12) that are placed in cylindrical fashion along the length of the electrode (8). Said moisture sensors (12) are adapted to detect liquid volume and/or pressure values in the local brain environment at the site of implantation. The liquid volume values are typically calibrated and scaled between two integral values, e.g. 100 and 1000, and measured in water fraction by volume (wfv) units. The liquid pressure values are measured in micro-pascals or milli-bars.

In the preferred embodiment of FIG. 2, one moisture sensor is placed on each side of each electrical contact (3). In this configuration, each pair of moisture sensors (12) is adapted to detect liquid volume and/or pressure values around each electrical contact (3). In these positions, the moisture sensors (12) provide a more precise indirect estimation regarding the position of any contact within the brain parenchyma by recording moisture values around each electrical contact. In addition, this configuration ascertains that the deviations in liquid volume and/or pressure are not due to a technical or manufacturing error. As a hypothetical example, a depth sEEG electrode (8) of the present invention is used to target the hippocampus, which is partially surrounded by ventricular space. If the moisture sensors of the most distal electrode contact indicate high liquid volume values (e.g. >500) and moderate liquid pressure values (e.g. around 50 uPa), it can be inferred that the depth sEEG electrode fell short of its intended target and needs to be advanced to reach hippocampal space. When the electrode advancement is performed, the moisture sensors of the 3^{rd} or 4^{th} most distal electrical contacts are expected to show elevated liquid volume values (e.g. before around 300-500, after >500), as well as liquid pressure values (e.g. before around 10 uPa, after around 50 uPa), suggesting that the depth sEEG electrode (8) is in its intended position. In another scenario, the same depth sEEG electrode (8) is used to target the hippocampus; if it shows stably elevated liquid volume values (e.g. >500) in all moisture sensors after implantation, without considerable rise in liquid pressure values (e.g. around 10 uPa), it can be inferred that the depth sEEG electrode (8) resides in subdural space. In a similar scenario, the electrode may show stably reduced liquid volume values in all moisture sensors (e.g. around 100-200), from which it can be inferred that the depth sEEG electrode (8) resides in extra-dural space. In both cases, the depth sEEG electrode (8) needs to be removed and a new dural/parenchymal pathway has to be formed before re-implantation. In another hypothetical example, the same depth sEEG electrode (8) is used to target the hippocampus; after implantation it shows progressively elevating liquid volume values (e.g. initially around 300, then around 500, and soon after saturating at 1000) and liquid pressure values (e.g. initially around 50 uPa, then around 100 uPa, and soon after >200 uPa) in the moisture sensors of the most proximal/superficial contacts. This scenario infers the establishment of a dural, subdural or intra-parenchymal hemorrhage, which constitutes a medical emergency and has to be addressed accordingly.

According to one preferred embodiment, the electrical contacts (3) are uniformly distributed along the length of the electrode. The distance between successive contacts is preferably from 2 to 4 cm. The temperature sensors (5) are placed between the electrical contacts (3) in such a way that the distance between successive contacts is partially covered by the temperature sensors (5). In a more preferred embodiment, one moisture sensor (12) is placed on each side of each electrical contact (3) in such a way that the distance between successive contacts is partially covered by the moisture sensors (12). For example, the temperature sensors (5) may cover 50% of the distance between successive electrical contacts (3), aligned in the middle, and the moisture sensors (12) may cover 25% each, aligned on each side of the electrical contacts (3).

In an alternative embodiment, the one or more moisture sensors (12) are placed at or near the distal tip of the electrode (8). In a further alternative embodiment, the one or more moisture sensors (12) are placed at or near the center of the electrode (8). In fact, various electrode embodiments can have only one moisture sensor or any other number of moisture sensors that are placed anywhere on the electrode in locations that are not related to the locations of the contacts (3).

The interior of the depth electrode consists of a) the fine wiring (6) of the standard electrical contacts (3) through which the intracranial neuronal electrical stimulation is conducted, b) the unidirectional fine wiring (7) of the temperature sensors (5) which convert the local temperature values into analog form, and c) the unidirectional fine wiring (13) of the moisture sensors (12) which convert the local moisture values into analog form. All of the fine wiring inside the proposed deep-reinforced electrode (8) are led to the proximal end of the electrode where they can be connected to the respective neurostimulation or sEEG device. The thickness of the fine wiring inside the deep electrode, for all standard electrical contacts (6), temperature sensors (7) and moisture sensors (12), is preferably of the order of 0.1 mm. The interior surface of depth sEEG electrodes is typically covered with an insulation layer, within which all fine wiring is channeled for the purpose of ensuring good signal conductivity and avoiding short-circuits with other sensors and/or wiring elements. The internal surface insulation layer can be made up for any known biocompatible insulation material, such as liquid crystal polymer, parylene, polyimide.

According to one embodiment, the standard electrical contacts (3) are made of a biocompatible metal, such as platinum. The corresponding leads (6, 7 and 13) are also made of biocompatible conductive material, such as platinum, copper, silver, or other alloys. The temperature sensors (5) are thermistors or thermocouples or resistance temperature detectors, covered by a biocompatible membrane with properties of good temperature conduction, non-absorption of liquids, and a high degree of hydrolytic stability. It is understood that other temperature sensors can also be used. The moisture sensors (12) are biocompatible capacitive or resistive elements, covered by a biocompatible membrane with good liquid absorption properties and a high degree of hydrolytic stability. Other biocompatible moisture sensors may also be used.

The cylindrical body of the electrode (2), on which the electrical contacts (3), the temperature sensors (5) and the moisture sensors (12) are placed and through which their wiring (6, 7, 13) passes, is preferably made of elastic biocompatible plastic, such as polyvinyl chloride (PVC).

In a preferred embodiment, in addition to delivery of intracranial neuronal electrical stimulation, the electrical contacts (3) are further adapted to record electrical brain signals. The electrical contacts (3) of this embodiment may be connected to an external neurostimulation or sEEG device via bi-directional fine wiring (6) which records the electrical signals of the brain and through which the intracranial neuronal electrical stimulation is conducted.

As one skilled in the relevant art would find apparent from the above description, the arrangement illustrated in FIG. 2 is only one example of the present invention. Many other configurations or modifications are possible, as discussed in the present disclosure and as could be made by one skilled in the art without departing from the scope of the invention as defined by the claims.

In a preferred embodiment, the temperature sensors (5) of the proposed depth sEEG electrode (8) can be connected to an external electrical device, preferably an electrical neurostimulation device, in such a way that, when the electrode is in operation and immediately after the onset of an intracranial neuronal electrical stimulation and throughout its duration, the values of the local temperature are transferred in analog form to the neurostimulation device. According to an even more preferred embodiment, when the temperature value detected by one or more temperature sensors (5) of the electrode rises above predetermined safety limits, the external electrical neurostimulation device immediately interrupts the supply of intracranial neuronal electrical stimulation in order to stop the ongoing neurostimulation and to protect the brain parenchyma (1) from side effects of thermal nature.

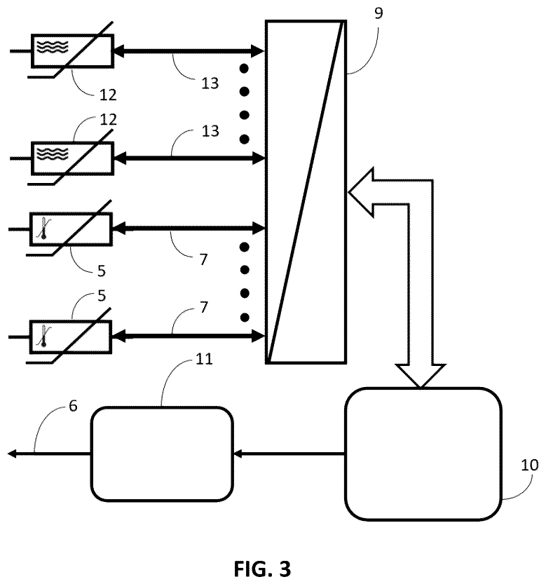

According to the configuration suggested in FIG. 3, when the electrode is in operation and connected to the electrical neurostimulation device, the analog temperature values transmitted by the temperature sensors (5) through their dedicated internal wiring (7) are led to a digitizer (analog signal to digital converter) (9) within the neurostimulation device. The digitizer, based on the analog input temperature signal, produces at its output a calibrated digital temperature value corresponding to each temperature sensor (5). The respective digital temperature values are transferred to the central unit or other peripheral processing unit (10), where they are compared as to whether they remain within the predetermined safety limits. As long as all temperature values remain within the predetermined safety limits for healthy brain tissue composition, the central or peripheral processing unit (10) allows the neurostimulation unit (11) to continue delivering intracranial neuronal electrical stimulation through the wiring of the standard electrical contacts (6). If one or more of the temperature values rise above and outside the predetermined safety limits, the central or peripheral processing unit (10) immediately interrupts the operation of the neurostimulation unit (11) in order to stop the ongoing intracranial neuronal electrical stimulation and protect the brain tissue (1) from side effects of a thermal nature.

In a preferred embodiment, the moisture sensors (12) of the proposed depth sEEG electrode (8) are connected to an external electrical device, preferably an electrical neurostimulation or sEEG device, in such a way that, when the electrode is in operation and at regular intervals after the electrode's (8) implantation in the brain parenchyma (1), all local liquid volume and/or pressure values are transferred in analog form to the neurostimulation or sEEG device. When the liquid volume and/or pressure values in all sensors are stable and elevated beyond intra-parenchymal levels, this suggests that the depth sEEG electrode (8) has been mislocated outside the brain parenchyma (1), into subdural space, and should be removed for re-implantation. When the liquid volume and/or pressure in all sensors is stable, with the liquid volume values reduced below intra-parenchymal levels and the liquid pressure values low, this suggests that the depth sEEG electrode (8) has been mislocated outside the brain parenchyma (1), into extradural space, and should also be removed for re-implantation. When the liquid volume and/or pressure values in one or few adjacent sensors are progressively elevated beyond intra-parenchymal levels, this suggests the establishment of a subdural or intra-parenchymal hemorrhage, which constitutes a medical emergency and has to be addressed accordingly. According to the configuration suggested in FIG. 3, when the electrode is in operation and connected to the electrical neurostimulation or sEEG device, the analog liquid volume and/or pressure values transmitted by the moisture sensors (12) through their dedicated internal wiring (13) are led to a digitizer (analog signal to digital converter) (9) within each neurostimulation or sEEG device. The digitizer, based on the analog input moisture signal, produces at its output calibrated digital liquid volume and/or pressure values corresponding to each moisture sensor (5). The respective digital liquid volume and/or pressure values are transferred to the central unit or some other peripheral processing unit (10), where they are compared as to whether they remain within the baseline levels. These values are conveyed to a graphic user interface, where the physician/neurosurgeon can evaluate the liquid volume and/or pressure values and assess the location of the implanted electrode, as well as the risk of intracranial hemorrhage, in order to act accordingly.

In certain embodiments, the external electrical device is a neurostimulation device. In other embodiments, the external electrical device is an sEEG device, that records the electrical signals of the brain through depth sEEG electrodes. In the latter embodiments, the electrode (8) may initially be used in the diagnostic sEEG phase for determining the area of onset of seizures. The signal(s) received by the moisture sensor(s) (12) help guide the physician/surgeon to correctly implant the electrode (8). Once the diagnostic sEEG phase is completed and the area of seizure onset has been determined, the physician may leave the electrode inside the brain, disconnect it externally from the sEEG device and connect it directly to the neurostimulation device, so that the same electrode may be also be used for neurostimulation in the therapeutic phase. During neurostimulation, the electrode is configured so that the signal(s) received by the temperature sensor(s) (5) are also transferred to the neurostimulation device; if the temperature value detected by one or more temperature sensors (5) rises above predetermined safety limits, the intracranial neuronal electrical stimulation is immediately interrupted.

In a more preferred embodiment, the one or more temperature sensors (5) and the one or more moisture sensors (12) of the proposed depth sEEG electrode (8) are connected to an external electrical device, preferably an electrical neurostimulation device. Said sensors are connected to the external electrical device as explained above, such that, if the temperature values are elevated beyond predetermined safety limits, the external device immediately interrupts the intracranial neuronal electrical stimulation; if the liquid volume and/or pressure values from one or more moisture sensors indicate that one or more contacts reside in cerebrospinal fluid space inside or outside the brain parenchyma, the electrode is removed for re-implantation; and if the liquid volume and/or pressure values from one or more moisture sensors indicate a potential subdural or intra-parenchymal hemorrhage, early action to stop the hemorrhage can be taken.

Also provided is an electrical device, such as an electrical neurostimulation or sEEG device, incorporating one or more electrodes (8) as disclosed herein. In certain embodiments, said device is designed to interrupt the intracranial neuronal electrical stimulation when one or more temperature sensors (5) register a local increase in temperature above predetermined safety limits. The electrical neurostimulation device includes a neurostimulation unit (11) for providing intracranial neuronal electrical stimulation. It may also include a processing unit (10) which receives the temperature values detected by the temperature sensors (5) and compares them with predetermined temperature values. If one or more of the temperature values rise above and outside the predetermined safety limits, the central or peripheral processing unit (10) immediately interrupts the operation of the neurostimulation unit (11) in order to stop the ongoing intracranial neuronal electrical stimulation and protect the brain parenchyma (1) from side effects of a thermal nature. Said device may also optionally include a digitizer (analog to digital converter) (9) which, at regular intervals determined by the specifications of the neurostimulation device, based on the input analog temperature signal, produces at its output a calibrated digital temperature value corresponding to each temperature sensor (5).

In other embodiments, said device is designed to receive the liquid volume and/or pressure values along the one or more electrical contacts (3) of each implanted electrode (8) and display them for the physician/neurosurgeon responsible for the sEEG procedure, in order for him/her to evaluate the liquid volume and/or pressure values and assess the location of the implanted electrode, as well as the risk of intracranial hemorrhage, so as to act accordingly. The electrical neurostimulation or sEEG device includes a processing unit (10) which receives the moisture values detected by the moisture sensors (12), where they are compared as to whether they remain within the baseline levels. Said device may also optionally include a digitizer (analog to digital converter) (9) which, at regular intervals determined by the specifications of the neurostimulation or sEEG device, based on the input analog moisture signal, produces at its output a graded digital moisture value corresponding to each moisture sensor (5).

In most preferred embodiments, said device is designed to interrupt the intracranial neuronal electrical stimulation when one or more temperature sensors (5) register a local increase in temperature above predetermined safety limits and to receive the moisture values along the one or more electrical contacts (3) of each implanted electrode as described above.

In aspects, the device disclosed herein may be used for a range of applications, some non-limiting examples of which include intracranial diagnostic and/or therapeutic procedures for patients with epilepsy, Parkinson's disease (PD), essential tremor, dystonia, obsessive-compulsive disorder, or other neurological diseases or movement disorders. Additional uses and details thereof are described throughout the present disclosure.

1. A depth stereo-electroencephalography (sEEG) electrode (8) configured to be implanted into the brain parenchyma (1), comprising:
a series of electrical contacts (3) along the length of the electrode configured to provide intracranial neuronal electrical stimulation (4);
one or more temperature sensors (5) interposed between one or more electrical contacts (3), wherein each temperature sensor (5) is adapted to detect the temperature values generated in the brain parenchyma (1) by the generated electrical field (4) as a result of prolonged and/or chronic intracranial neuronal electrical stimulation; and
one or more moisture sensors (12), wherein each moisture sensor (12) is adapted to detect the liquid volume and/or pressure values at the site of implantation, in order to detect deviations from baseline intra-parenchymal liquid volume and/or pressure values.

2. The electrode according to claim 1, wherein one temperature sensor (5) is placed between each two successive contacts (3), and wherein each temperature sensor (5) is adapted to detect the temperature values generated in the brain parenchyma (1) between adjacent electrical contacts.

3. The electrode according to claims 1 or 2, wherein one moisture sensor (12) is placed on each side of each electrical contact (3) and wherein each moisture sensor (12) is adapted to detect the liquid volume and/or pressure values around each electrical contact (3).

4. The electrode according to anyone of claims 1 to 3, wherein the electrical contacts (3) are further configured to record the electrical brain signals.

5. The electrode according to anyone of claims 1 to 4, which further comprises means (7) for electrically connecting the temperature sensor(s) (5) to an external electrical device, so that, when the electrode is in operation, the signal(s) received by the temperature sensor(s) (5) are or can be transferred to the external device.

6. The electrode according to anyone of claims 1 to 5, which further comprises means (13) for electrically connecting the moisture sensor(s) (12) to an external electrical device, so that, when the electrode is in operation, the signal(s) received by the moisture sensor(s) (12) are transferred to the external device.

7. The electrode according to claim 5 or 6, wherein the external device is an electrical neurostimulation device.

8. The electrode according to claim 7, which is configured so that, when the temperature value detected by one or more temperature sensors (5) rises above predetermined safety limits, the intracranial neuronal electrical stimulation is immediately interrupted.

9. The electrode according to claim 8, which is configured so that, when the temperature value detected by one or more temperature sensors (5) of the electrode (8) rises above predetermined safety limits, the external electrical neurostimulation device immediately interrupts the supply of intracranial neuronal electrical stimulation to the electrical contacts (3) of the electrode.

10. The electrode according to claim 6, wherein the external device is an electrical sEEG device.

11. The electrode according to anyone of the previous claims, which is configured so that, deviations of the liquid volume and/or pressure values detected at the site of implantation from baseline intra-parenchymal liquid volume and/or pressure values are indicative of electrode mislocation or potential intracranial hemorrhage.
